(19) United States
(12) Patent Application Publication
(10) Pub. No.: US 2016/0299081 A1
(43) Pub. Date: Oct. 13, 2016

(54) BIO-ENABLED PLASMONIC SUPERSTRUCTURES WITH BUILT-IN AND ACCESSIBLE HOTSPOTS

(71) Applicant: Washington University in St. Louis, St. Louis, MO (US)

(72) Inventors: Srikanth Singamaneni, St. Louis, MO (US); Limei Tian, St. Louis, MO (US)

(21) Appl. No.: 15/096,505

(22) Filed: Apr. 12, 2016

Related U.S. Application Data
(60) Provisional application No. 62/146,568, filed on Apr. 13, 2015.

(51) Int. Cl. G01N 21/65; A61K 9/51; A61K 41/00; G01N 33/574; G01N 33/58

(52) U.S. Cl. CPC G01N 21/658; G01N 33/57438; G01N 33/587; A61K 41/0052; A61K 9/5192; A61K 9/5146; A61K 9/5115

(57) ABSTRACT

The present disclosure relates generally to plasmonic superstructures having a nanostructure core and a plurality of nanoparticle satellites and methods for preparing plasmonic superstructures. The present disclosure is further directed to methods of bioimaging, biosensing and therapeutic applications using the plasmonic superstructures.

### CROSS-REFERENCE TO RELATED APPLICATIONS

This application claims priority from U.S. Ser. No. 62/146,568, filed Apr. 13, 2015, the disclosure of which is hereby incorporated by reference in its entirety.

### STATEMENT REGARDING FEDERALLY SPONSORED RESEARCH & DEVELOPMENT

This disclosure was made with government support under grant CBET-1254399, awarded by the National Science Foundation. The U.S. Government has certain rights in the invention.

### FIELD OF THE DISCLOSURE

The present disclosure relates generally to plasmonic superstructures for bioimaging, biosensing and therapeutic applications. More particularly the present disclosure is directed to plasmonic superstructures having a nanostructure core and a plurality of nanoparticle satellites, methods for preparing plasmonic superstructures and methods of using plasmonic superstructures for bioimaging, biosensing and therapeutic applications.

### BACKGROUND OF THE DISCLOSURE

Surface enhanced Raman scattering (SERS) represents a powerful bioimaging modality for image-guided interventions in intraoperative settings. Conventional contrast agents, often termed SERS probes, are comprised of individual plasmonic nanostructures or lightly aggregated or assembled plasmonic nanostructures, which suffer from either poor brightness, complex synthesis or lack of stability in complex biological milieu. The contribution of a relatively small number of electromagnetic (EM) hotspots (63 out of 106 active sites) can be quite significant (—25%) in the overall SERS signal. To date, very few SERS probes based on individual nanostructures host built-in EM hotspots. For example, trapping Raman reporters between a plasmonic core and a shell that are separated by a sub-nanometer gap causes large enhancement of Raman signals from the reporter molecules. However, the EM hotspots in such core-shell nanostructures are not accessible for the surrounding biological environment, thus limiting them to simple structure contrast agents. Accordingly, there exists a need for plasmonic nanoconstructs with built-in and accessible EM hotspots for functional molecular bioimaging such as sensing a specific (bio)chemical stimulus and molecular process.

### BRIEF DESCRIPTION OF THE DISCLOSURE

The present disclosure relates generally to plasmonic superstructures for bioimaging, biosensing and therapeutic applications. More particularly the present disclosure is directed to plasmonic superstructures having a nanostructure core and a plurality of nanoparticle satellites, methods for preparing plasmonic superstructures and methods of using plasmonic superstructures for bioimaging, biosensing and therapeutic applications.

In one aspect, the present disclosure is directed to a plasmonic superstructure. The plasmonic superstructure comprises a nanostructure core; a polymer coating the nanostructure core; and a plurality of nanoparticle satellites coupled to the nanostructure core.

In another aspect, the present disclosure is directed to a method of preparing a plasmonic superstructure. The method comprises: preparing a nanostructure core; modifying the nanostructure core with a polyanion to prepare a polyanion layer on the nanostructure core; incubating the nanostructure core with the polyanion layer in a polymer solution; incubating the nanostructure core with a metal nanoparticle satellite growth solution, wherein a plurality of nanoparticles form a plurality of nanoparticle satellites coupled to the nanostructure core to form the plasmonic superstructure comprising a nanostructure core and a plurality of nanoparticle satellites.

In another aspect, the present disclosure is directed to a method of measuring intracellular pH using a plasmonic superstructure. The method comprises: contacting a cell with a solution comprising a plasmonic superstructure, wherein the plasmonic superstructure comprises a nanostructure core; a polymer; and a plurality of nanoparticle satellites coupled to the nanostructure core; incubating the cell for a sufficient time to allow for internalization of the plasmonic nanostructure; exciting the plasmonic nanostructure using an excitation source; and analyzing the cell.

In another aspect, the present disclosure is directed to a method of photothermal therapy. The method comprises: contacting a cell with a solution comprising a plasmonic superstructure, wherein the plasmonic superstructure comprises a nanostructure core; a polymer; and a plurality of nanoparticle satellites coupled to the nanostructure core; incubating the cell with a plasmonic superstructure for a sufficient time to allow for internalization of the plasmonic superstructure; and irradiating the cell.

In accordance with the present disclosure, compositions and methods have been discovered that surprisingly allow for bioimaging, biosensing and therapeutic applications using the compositions. The methods of the present disclosure have a broad and significant impact, as they provide a universal method to realize size- and shape-controlled plasmonic superstructures for bioimaging, biosensing and therapeutic applications that were previously unidentifiable using traditional methods using core-shell nanostructures having Raman reporters trapped between a plasmonic core and a shell that are not accessible for the surrounding biological environment, and are thus limited to simple structure contrast agents.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The disclosure will be better understood, and features, aspects and advantages other than those set forth above will become apparent when consideration is given to the following detailed description thereof. Such detailed description makes reference to the following drawings, wherein:

FIG. 1A is a schematic illustration depicting the synthesis of AuNPR plasmonic superstructures having Au nanoparticle satellites on a Au nanorod (AuNR) core by modifying the gold nanorod (AuNR) with a biopolymer, poly-L-histidine (poly-his), and subsequent growth of Au nanoparticle satellites on the AuNR surface.

FIG. 1B is a graph depicting normalized extinction spectra of AuNR, poly-his modified AuNR before and after binding Au³⁺ ions, and AuNPR showing the progressive red shift in localized surface plasmon resonance (LSPR) wavelength due to increase in effective refractive index during surface modification and plasmon coupling after the growth of Au nanoparticle satellites.

FIG. 1C is a representative transmission electron microscopy (TEM) image of AuNR cores.

FIG. 1D is a representative TEM image of AuNPR showing the uniform growth of Au nanoparticle satellites on the AuNR cores.

FIG. 1E is a graph depicting normalized extinction spectra of AuNS and AuNPS showing the red shift in LSPR wavelength due to the plasmon coupling between the Au nanoparticle satellites and the nanostructure core.

FIG. 1F is a representative TEM image of AuNS cores.

FIG. 1G is a representative TEM image of AuNPS showing the uniform growth of Au nanoparticle satellites on the AuNS cores.

FIG. 2A is a high resolution TEM (HRTEM) image showing the sub-3 nm interstices between the nanoparticle satellites in an AuNPR, which provide built-in electromagnetic hotspots and large SERS activity.

FIG. 2B is a graph depicting the average SERS spectra obtained from AuNR and AuNPR solution following the adsorption of Raman reporters (p-mercaptobenzoic acid) on the surface of the nanostructures. Inset shows finite difference time domain (FDTD) simulations confirming the EM hotspots of the plasmonic superstructures.

FIG. 2C is a high resolution TEM (HRTEM) image showing the sub-3 nm interstices between the nanoparticle satellites in an AuNPS, which provide built-in electromagnetic hotspots and large SERS activity.

FIG. 2D is a graph depicting the average SERS spectra obtained from AuNS and AuNPS solution following the adsorption of Raman reporters (p-mercaptobenzoic acid) on the surface of the nanostructures. Inset shows finite difference time domain (FDTD) simulations confirming the EM hotspots of the plasmonic superstructures.

FIG. 2E is a graph depicting representative SERS spectra from AuNPR at pH 5 and 9, showing the increase in the ratio of relative Raman intensity of symmetric carboxylate stretching band of p-mercaptobenzoic acid at 1394 cm⁻¹ to 1079 cm⁻¹ with increasing pH due to the deprotonation of the carboxylate group.

FIG. 2F is a graph depicting a pH calibration plot showing the variation of the ratio of intensity of Raman bands at 1394 cm⁻¹ to 1079 cm⁻¹ with external pH.

FIG. 3A is schematic illustrating the endocytosis, intracellular transportation and exocytosis of AuNPRs. AuNPRs enter the cell by receptor-mediated endocytosis and non-specific micropinocytosis. After internalization, the intravesicular pH drops along the endocytic pathway, from pH 6.0-6.5 in early endocytic vesicles to pH 4.5-5.5 in multivesicular bodies and multilamellar lysosomes. The AuNPR preserve their highly developed core-satellite structure even after internalizing into the cell, which allows for maintenance of their high SERS activity.

FIG. 3B is a transmission electron micrograph (TEM) image of AuNPRs entering a 786-O human renal adenocarcinoma cell by receptor-mediated endocytosis and non-specific micropinocytosis.

FIG. 3C is a transmission electron micrograph (TEM) image of AuNPRs in an early endocytic vesicle of a 786-O human renal adenocarcinoma cell.

FIG. 3D is a transmission electron micrograph (TEM) image of AuNPRs in multivesicular body of a 786-O human renal adenocarcinoma cell.

FIG. 3E is transmission electron micrograph (TEM) image of AuNPRs in multilamellar lysosome of a 786-O human renal adenocarcinoma cell.

FIG. 4A depicts SERS intensity maps of 1079 cm⁻¹ Raman band of pMBA at different time points (0 minutes, 30 minutes, and 60 minutes) showing the clear delineation of the shape of the cell spread on a quartz substrate.

FIG. 4B depicts histograms analyzed from the SERS intensity maps in FIG. 4A at different time points (0 minutes, 30 minutes, and 60 minutes) showing ˜10% mean intensity drop after every 30 minutes. Insets depict images using a dark field optical microscope to locate the cell and match the cell margin with the SERS intensity maps.

FIG. 4C depicts the spatiotemporal pH maps analyzed from the intensity ratio of Raman bands 1394 cm⁻¹/1079 cm⁻¹ at different time points (0 minutes, 30 minutes, and 60 minutes) based on the pH calibration curve at each pixel color-coded using MATLAB.

FIG. 4D depicts time-dependent pH histograms at different time points (0 minutes, 30 minutes, and 60 minutes) showing the decrease in the number of pixels corresponding to physiological pH 7.0-7.5 from 49% to 38% and increase in the number of pixels corresponding to acid pH 4.5-5.5 from 16% to 32%, which visualize the intravesicular pH drop in the endosomal maturation process.

FIG. 5A are infrared images depicting the rise in the temperature of water, AuNPS and AuNPR upon irradiation with NIR laser (λₑₓ=808 nm) at a power density of 0.3 W/cm².

FIG. 5B is a graph depicting the significantly higher rise in temperature of AuNPR solution (ΔT=24° C.) compared to AuNPS (ΔT=8° C.) and water (ΔT=<1° C.) upon irradiation with NIR laser.

FIG. 5C is a graph depicting cell viability following the NIR laser irradiation of control cells and cells incubated with AuNPS and AuNPR for different time durations as quantified by MTT assay. While control cells and cells incubated with AuNPS exhibited high viability consistent with the small rise in temperature, cells incubated with AuNPR exhibited significant reduction in viability.

FIG. 5D are images of control cells and cells incubated with AuNPS and AuNPR. Following the irradiation with NIR laser (λₑₓ=808 nm), control cells and cells incubated with AuNPS exhibited green fluorescence (indicating live cells) while cells incubated with AuNPR exhibited red fluorescence (indicating dead cells). Top row are bright field images; middle row are green fluorescent images; bottom row are red fluorescent images.

FIG. 6A is a graph depicting the zeta potential following each step of AuNPR synthesis.

FIG. 6B is a graph depicting Raman spectra of AuNR modified with PSS and poly-L-histidine and the bulk form of PSS and poly-L-histidine.

FIG. 7A is a TEM image of AuNR.

FIG. 7B is a TEM image of AuNPRs synthesized without poly-L-histidine.

FIG. 7C is a TEM image of AuNPRs synthesized with poly-L-histidine.

FIG. 8A is a graph depicting extinction spectra of AuNPRs synthesized with 10 μl-60 μl HAuCl₄ precursor solution.

FIG. 8B is a TEM image of AuNPRs synthesized with 10 μl HAuCl₄ precursor solution.

FIG. 8C is a TEM image of AuNPRs synthesized with 20 μl HAuCl₄ precursor solution.

FIG. 8D is a TEM image of AuNPRs synthesized with 30 μl HAuCl₄ precursor solution.

FIG. 8E is a TEM image of AuNPRs synthesized with 40 μl HAuCl₄ precursor solution.

FIG. 8F is a TEM image of AuNPRs synthesized with 60 μl HAuCl₄ precursor solution.

FIG. 9A is a TEM image of AuNPRs synthesized with 1 M HCl to achieve the reaction at pH 2.

FIG. 9B is a TEM image of AuNPRs synthesized without HCl or NaOH to achieve the reaction at pH 3.4.

FIG. 9C is a TEM image of AuNPRs synthesized with 0.1 M NaOH to achieve the reaction at pH 6.4.

FIG. 10A is a graph depicting extinction spectra of AuNPR before and after chemisorption of pMBA showing a 5 nm red shift in the longitudinal LSPR wavelength upon adsorption of pMBA.

FIG. 10B is a graph depicting SERS spectra of AuNPR synthesized using different amounts of HAuCl₄ precursor solutions.

FIG. 10C is a histogram depicting the intensity of 1079 cm⁻¹ Raman band for AuNR, AuNPRs, and AuNPSs synthesized using different amounts of HAuCl₄ precursor solutions.

FIG. 10D is a graph depicting representative SERS spectra from AuNPR at pH 5 to 9 showing the relative Raman intensity of symmetric carboxylate stretching band of pMBA at 1394 cm⁻¹ with respect to 1079 cm⁻¹ increase with increasing pH due to the deprotonation of the carboxylate group.

FIG. 11A is a graph depicting extinction spectra of AuNPR dispersed in 10% fetal bovine serum (FBS) at different time points showing the stability of the plasmonic superstructures in complex biological milieu.

FIG. 11B is a histogram depicting percent viability of human renal adenocarcinoma cells (786-O cells) incubated with AuNPR-pMBA at different concentrations of Au atoms showing the minimal toxicity of the plasmonic superstructures.

FIG. 11C is a histogram depicting percent viability of human renal adenocarcinoma cells (786-O cells) incubated with AuNPS-pMBA at different concentrations of Au atoms showing the minimal toxicity of the plasmonic superstructures.

FIG. 11D is a histogram depicting percent viability of renal primary proximal tubule epithelial cells (RPTC) incubated with AuNPR-pMBA at different concentrations of Au atoms showing the minimal toxicity of the plasmonic superstructures.

FIG. 11E is a histogram depicting percent viability of renal primary proximal tubule epithelial cells (RPTC) incubated with AuNPS-pMBA at different concentrations of Au atoms showing the minimal toxicity of the plasmonic superstructures.

FIG. 12A is a bright field image of cells incubated with AuNPR to ensure the viability of cells.

FIG. 12B is a green fluorescence image (indicating live cells) of cells incubated with AuNPR to ensure the viability of cells.

FIG. 13 is a graph depicting representative spectra at different pH as color-coded pixels using MATLAB®.

FIG. 14 is a graph depicting extinction spectra of AuNPR and AuNPS solution at the similar concentration of ˜55 μg/ml Au atoms.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the scope of the disclosure as defined by the appended claims.

### DETAILED DESCRIPTION OF THE DISCLOSURE

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

Plasmonic Superstructures

In one aspect, the present disclosure is directed to a plasmonic superstructure. The plasmonic superstructures include a nanostructure core; a polymer coating the nanostructure core; and a plurality of nanoparticle satellites coupled to the nanostructure core.

The plasmonic superstructures include a nanostructure core. The nanostructure core can be any shape. Suitable nanostructure core shapes can be, for example, rods, spheres, cubes, bipyramids, stars, and combinations thereof. Nanostructure cores can be, for example, gold nanostructure cores, silver nanostructure cores, copper nanostructure cores, aluminum nanostructure cores, and combinations thereof.

The plasmonic superstructures include a polymer coating the nanostructure core. Suitable polymers can be a biopolymer, a synthetic polymer, and combinations thereof. A particularly suitable biopolymer is poly-L-histidine. Particularly suitable synthetic polymers can be poly(allylamine hydrochloride) and poly(2-vinyl pyridine).

The plasmonic superstructures include a plurality of nanoparticle satellites. The nanoparticle satellites are formed by nanoparticles during the preparation of the plasmonic superstructures when a nanoparticle growth solution is introduced to polymer-coated nanostructure cores. Affinity of ions of the nanoparticle precursor with the polymer coating the nanostructure core facilitates the uniform nucleation and growth of nanoparticles to form a plurality of nanoparticle satellites coupled to the nanostructure core.

Nanoparticle satellites can be formed by incubating polymer-coated nanostructure cores with a metal growth solution. Particularly suitable metals to form nanoparticle satellites include gold, silver, copper, aluminum, and combinations thereof. For example, a gold growth solution made using HAuCl₄, for example, can be incubated with polymer-coated nanostructure cores. Capture of Au³⁺ ions by the polymer (such as imidazole groups of poly-L-histidine) results in the formation of a plurality of nanostructure satellite clusters on the nanostructure cores to form the plasmonic superstructures having the nanostructure cores and plurality of nanoparticle satellites. The size and areal density of the nanoparticle satellites on the nanostructure core can be tuned over a broad range by varying the amount of metal precursor in the growth solution. The concentration of the metal precursor in the growth solution can be from about 10 μM to about 150 μM. More particularly, the concentration of the metal precursor in the growth solution can be from about 20 μM to about 120 μM.

The plasmonic superstructures can further include a polyanion layer on the nanostructure cores. Particularly suitable polyanions can be, for example, poly(styrenesulfonate) (PSS), poly(acrylic acid), alginate, and combinations thereof. The polyanion layer provides stability to the nanostructure cores in a wide pH range and ionic strength range. The polyanion layer further provide for electrostatic interactions with the biopolymer.

The plasmonic superstructures can further include a protective layer. Suitable protective layers can be formed using hydrophilic polymers. Suitable hydrophilic polymers include thiol-modified polyethylene glycol, amine-terminated polyethylene glycol, carboxyl-terminated polyethylene glycol and combinations thereof.

The interstitial space between nanoparticles of the satellites can be about 3 nm. In other embodiments, the interstitial space between nanoparticles of the satellites is less than 3 nm. The interstitial space can be determined by measuring TEM images, for example.

Methods for Preparing Plasmonic Superstructures

In another aspect, the present disclosure is directed to a method for preparing plasmonic superstructures. The method includes preparing a nanostructure core; modifying the nanostructure core with a polyanion to prepare a polyanion layer on the nanostructure core; incubating the nanostructure core with the polyanion layer in a polymer solution; and incubating the nanostructure core with a metal nanoparticle satellite growth solution, wherein a plurality of nanoparticles form on the nanostructure core to form the plasmonic superstructure comprising a nanostructure core, a polymer, and a plurality of nanoparticle satellites.

Nanostructure cores can be prepared using a seed-mediated approach. The seed-mediated approach includes combining a seed solution with a growth solution and allowing the nanostructure cores to form. For example, gold nanorod cores can be prepared using a seed solution including sodium borohydride solution, cetyltrimethylammonium bromide (CTAB) and chloroauric acid (HAuCl₄) that is combined with a growth solution including CTAB, HAuCl₄, silver nitrate, ascorbic acid and hydrochloric acid (HCl). The combined solutions form gold nanorods. Gold nanorods can be analyzed by localized surface plasmon resonance (LSPR) and transmission electron microscopy (TEM). Gold nanosphere cores can also be prepared synthesized using a seed-mediated method in which a seed solution including cetyltrimethylammonium chloride (CTAC), HAuCl₄ and Sodium borohydride (NaBH₄) are combined with a growth solution including CTAC, HAuCl₄, and ascorbic acid. The combined solutions form gold nanospheres. The gold nanosphere cores can be analyzed by LSPR and TEM.

The nanostructure core can be any shape. Suitable nanostructure core shapes can be, for example, rods, spheres, cubes, bipyramids, stars, and combinations thereof. Nanostructure cores can be, for example, gold nanostructure cores, silver nanostructure cores, copper nanostructure cores, aluminum nanostructure cores, and combinations thereof.

In one embodiment, nanostructure cores are modified to have a polyanion layer. The polyanion layer provides stability to the nanostructure cores over a wide range of pH and ionic strength conditions. The polyanion layer surrounds or “coats” the outside surface of the nanostructure core. The polyanion layer is formed by incubating the nanostructure core in a polyanion solution. Nanostructure cores having the polyanion layer can be isolated from excess polyanion solution by centrifugation and washed with a buffer such as water, for example.

Particularly suitable polyanions can be, for example, poly(styrenesulfonate) (PSS), poly(acrylic acid), alginate, and combinations thereof.

In another embodiment, nanostructure cores are not modified to have a polyanion layer.

The method includes incubating the nanostructure core in a polymer solution. Suitable polymers can be a biopolymer, a synthetic polymer, and combinations thereof. A particularly suitable biopolymer is poly-L-histidine. Particularly suitable synthetic polymers can be poly(allylamine hydrochloride) and poly(2-vinyl pyridine)

After incubating the nanostructure core in a polymer solution, the method includes incubating the nanostructure core with a metal nanoparticle satellite growth solution, wherein a plurality of nanoparticle satellites form on the nanostructure core to form the plasmonic superstructure comprising a nanostructure core and a plurality of nanoparticle satellites.

The size and density of the nanoparticle satellites that form on the nanostructure core can be varied over a broad range by adjusting the amount of metal precursor in the growth solution. The size of nanoparticles that form nanoparticle satellites on the nanostructure core can be from about 5 nm to about 15 nm with interstitial spaces of from about 1 nm to about 3 nm. The size of the nanoparticle satellites can be measured using TEM, for example. The interstitial space between nanoparticles of the nanoparticle satellites can be measured using TEM, for example.

The pH of the combined seed solution and growth solution (i.e., the “reaction solution”) can be adjusted between pH 2 to pH 11 to control nucleation of the nanoparticles on the nanostructure core to form the nanoparticle satellites. A particularly suitable pH for nucleation of the nanoparticles on the nanostructure core to form the nanoparticle satellites is pH 6.4.

Growth of nanoparticles forming the nanoparticle satellites can be monitored by measuring LSPR wavelength and TEM, for example.

The method can further include incubating adding a solution including a reducing agent. Suitable reducing agents can be an aqueous ascorbic acid solution, hydroxylamine hydrochloride, sodium borohydride, formic acid, and combinations thereof.

The method can further include adding a solution including a capping agent. Suitable capping agents can be an aqueous polyvinylpyrrolidone solution, cetyltrimethylammonium bromide, cetyltrimethylammonium chloride, and combinations thereof.

The method can further include forming a protective layer on the plasmonic nanostructures. The protective layer is formed by incubating the plasmonic superstructure in a solution including methoxy polyethylene glycol thiol.

The method can further include adsorbing a Raman reporter to the plasmonic superstructures. The Raman reporter can be adsorbed to the plasmonic superstructure by incubating the plasmonic superstructure in a solution including a Raman reporter. Any suitable Raman reporter can be used. A particularly suitable Raman reporter is p-mercaptobenzoic acid (pMBA).

The method can further include incubating the plasmonic superstructure in serum. Incubating the plasmonic superstructure in serum can provide additional biocompatibility of the plasmonic superstructure. Incubating the plasmonic superstructure in serum can facilitate micropinocytosis and receptor-mediated endocytosis due to non-specific adsorption of serum proteins on the plasmonic superstructure surface. Any serum is suitable. Fetal bovine serum is particularly suitable. The plasmonic superstructure can be incubated in a solution comprising about 10% serum.

Methods of Measuring Intracellular pH Using Plasmonic Superstructures

In another aspect, the present disclosure is directed to a method of measuring intracellular pH using plasmonic superstructure. The method includes contacting a cell with a solution comprising a plurality of plasmonic superstructure, wherein the plasmonic superstructure comprises: a nanostructure core, a polymer, and a plurality of nanoparticle satellites; incubating a cell with the solution for a sufficient time to allow for internalization of the plasmonic superstructure; exciting the plasmonic superstructure using an excitation source; and analyzing the cell.

Suitable methods for analyzing the cell can be, for example, Raman imaging. Raman imaging can be performed using a Raman microscope such as a confocal Raman microscope. Other suitable methods for analyzing the cell can be Dark-field scattering, two-photon photoluminescence, fluorescence, and combinations thereof.

Intracellular pH can be determined by analyzing the intensity ratio of Raman bands at 1394 cm⁻¹ and 1079 cm⁻¹.

A particularly suitable plasmonic superstructure for bioimaging is a plasmonic superstructure including a gold nanosphere core and a plurality of gold nanoparticle satellites.

Method of Photothermal Therapy

In another aspect, the present disclosure is directed to a method of photothermal therapy. The method includes contacting a cell with a solution comprising a plasmonic superstructure, wherein the plasmonic superstructure comprises: a nanostructure core; a polymer, and a plurality of nanoparticle satellites; incubating a cell with the solution for a sufficient time to allow for internalization of the plasmonic superstructure; and irradiating the cell.

A particularly suitable method for irradiation includes irradiation with 808 nm laser (at a power density of 0.3 W/cm²). Irradiation can occur for any desired time duration.

A particularly suitable plasmonic nanostructure for photothermal therapy is a plasmonic superstructure including a gold nanorod core and a plurality of gold nanoparticle satellites.

### EXAMPLES

### Materials

Unless specified, all the chemicals were purchased and used without further purification. Cetyltrimethylammoniumbromide (CTAB), chloroauric acid, ascorbic acid, sodium borohydride, poly(styrene sulfonate) (PSS) (Mw=70,000 g/mol), poly-L-histidine hydrochloride (Mw>5,000 g/mol), sodium hydroxide (NaOH), mercaptobenzoic acid (MBA), penicillin/streptomycin, and G418 sulfate were purchased from Sigma-Aldrich. Silver nitrate and Lonza RPMI-1640 with 25 mM HEPES and L-Glutamine was purchased from VWR International. Methoxy PEG thiol (SH-PEG, Mw=5,000 g/mol) was purchased from Jenkem Technology. Phosphate and acetate buffer from pH 5.0 to 9.0 were purchased from G-Biosciences. Human renal cancer cell line (786-O) and Renal Proximal Tubule Cells (RPTEC) were purchased from ATCC (Manassas, Va.). Fetal bovine serum (FBS), Trypsin-EDTA (0.25%), and Dulbecco's Phosphate-Buffered Saline (DPBS) were purchased from Life Technologies.

Characterization

TEM images were obtained using either field emission TEM (JEM-2100F, JEOL) or JEOL 2010 LaB6 operating at an accelerating voltage of 200 kV. UV-vis extinction spectra were collected using a Shimadzu 1800 spectrophotometer. Zeta potential measurements were performed using MalvernZetasizer (Nano ZS). Raman spectra were collected using a Renishaw in Via confocal Raman spectrometer mounted on a Leica microscope with 20× objective (NA=0.40) in the range of 600-1800 cm⁻¹ with one accumulation and 10 second exposure time. A 785 nm wavelength diode laser coupled to a holographic notch filter was used to excite the sample.

Synthesis of Gold Nanorods (AuNRs)

Gold nanorods (AuNRs) were synthesized using a seed-mediated approach. Seed solution was prepared by adding 0.6 ml of an ice-cold sodium borohydride solution (10 mM) into 10 ml of 0.1 M cetyltrimethylammonium bromide (CTAB) and 2.5×10⁻⁴M chloroauric acid (HAuCl₄) solution under vigorous stirring at room temperature. The color of the seed solution changed from yellow to brown. Growth solution was prepared by mixing 100 ml of CTAB (0.1 M), 5 ml of HAuCl₄ (10 mM), 1.0 ml of silver nitrate (10 mM), 0.8 ml of ascorbic acid (0.1 M) and 1 ml of HCl (1M) consecutively. The solution was homogenized by gentle stirring. To the resulting colorless solution, 0.24 ml of freshly prepared seed solution was added and set aside in the dark for 14 hours.

Synthesis of Gold Nanospheres (AuNSs)

Gold nanospheres (AuNSs) were synthesized using a seed-mediated method. A seed solution was prepared by vigorous mixing of 9.5 ml of aqueous cetyltrimethylammonium chloride (CTAC) solution (0.1M) and 515 μl of aqueous HAuCl₄ solution (4.86 mM), with 450 μl of ice-cold NaBH₄. The seed solution was aged for 1 hour at 30° C. in a hot bath. In the next step, growth solution was prepared by mixing 9.5 mL of aqueous CTAC solution (0.1M), 515 μl of aqueous HAuCl₄ solution (4.86 mM), and 150 μl of ascorbic acid (0.04 M). To this colorless solution, 25 μl of seed was added with vigorous stirring and kept undisturbed for two days to obtain highly uniform spherical nanospheres with LSPR peak at 530 nm. The shape and uniformity of the nanospheres were verified by TEM.

Preparation of Polyelectrolyte-Coated Gold Nanorods (AuNRs) and Gold Nanospheres (AuNSs)

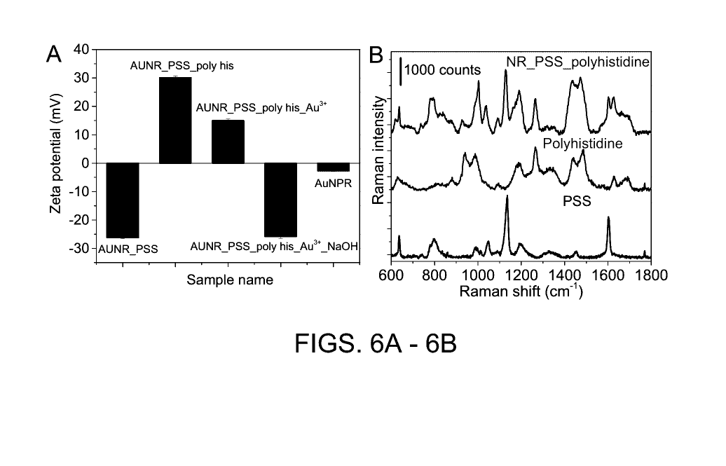

To prepare polyelectrolyte-coated gold nanorods (AuNRs) and gold nanospheres (AuNSs), 1 ml of a twice centrifuged AuNRs solution or a twice centrifuged AuNSs solution was added drop-wise to 0.5 ml of PSS solution (0.2% w/v) in 6 mM NaCl aqueous solution under vigorous stirring, followed by shaking for 3 hours. To remove excess PSS, the solution was centrifuged at 10,000 rpm for 10 minutes, and the pellet was dispersed in nanopure water after removing the supernatant. The surface charge of CTAB stabilized AuNRs, PSS coated AuNRs (AuNRs@PSS) were estimated by measuring the zeta potential of corresponding solution (FIGS. 6A & 6B).

Cell Viability Assay

To quantify the toxicity of nanomaterials, Methylthiazolyldiphenyl-tetrazolium bromide (MTT) assay was employed to probe the viability of 786-O cells incubated with various concentrations of PEGlated AuNPRs-MBA for 4 hours. 10 μl of 5 mg/ml methylthiazolyldiphenyl-tetrazolium bromide (MTT) in PBS was added to each well, followed by 4 hours of incubation. Then 100 μl of dimethyl sulfoxide (DMSO) was added to each well, including controls, followed by gentle swirl. The absorbance was measured at 570 nm using an Infinite F200 multimode reader (Tecan, Switzerland). Cell viability was normalized to that of 786-O cells cultured in the complete culture medium without the incubation with AuNPRs-MBA.

SERS Imaging of Live Cells

Human renal cancer cell line (786-O) was sub-cultured in RPMI-1640 medium with 10% fetal bovine serum (FBS) and antibiotics (100 g/ml penicillin/streptomycin) while the normal human RPTEC were cultured in the same media containing 100 μg/ml G418 Sulfate. Cells were grown in a water jacket incubator at 37° C. with 5% CO₂-humidified atmosphere in 25 cm² tissue culture flasks. Once the cells reached 90% confluence, they were washed with phosphate buffered saline (PBS) and detached with 2 ml of 0.25% trypsin-EDTA solution. After centrifugation, cells were dispersed in complete medium with 10% FBS and plated at a density of 1×10⁴ cells/cm² on a quartz substrate in a 35 mm flat-bottom culture dish. After overnight incubation at 37° C. with 5% CO₂-humidified atmosphere, 786-O cells were incubated with 3 ml of PEGylated AuNPRs-MBA dispersed in complete medium for 8 hours at 37° C. Then the cells were thoroughly rinsed with PBS twice to remove loosely bound AuNPRs on the cell surface and mounted on a live cell chamber with well controlled temperature and CO₂. After locating the cells using a dark-field microscope, the living cell imaging was performed using a confocal InVia Renishaw Raman microscope by collecting a 2D array of Raman spectra with 2 μm of spatial resolution using a 785 nm laser with 3 mW power using 20× objective and 3 second exposure time. A live/dead cell assay was performed to ensure the viability of cells after the living cell imaging (FIG. 13). Correspondingly, the cells were prepared the same way for TEM section imaging.

In Vitro Photothermal Therapy

The NIR irradiation was performed using an 808 nm wavelength diode laser for different durations and at a power density of 400 mW cm⁻². Following laser treatment, the cells were incubated with full medium for 16 hours and then stained with ethidium homobromide-1 and calcein AM dyes to produce green and red emission from live and dead cells, respectively. Methylthiazolyldiphenyl-tetrazolium bromide (MTT) assay was employed to probe the viability of 786-O cells. 10 μl of methylthiazolyldiphenyl-tetrazolium bromide (MTT) in PBS (5 mg/ml) was added to each well, followed by 4 hours of incubation. Subsequently, 100 μl of dimethyl sulfoxide (DMSO) was added to each well, including controls, followed by gentle swirl. The absorbance was measured at 570 nm using an Infinite F200 multimode reader (Tecan, Switzerland). Cell viability was normalized to that of 786-O cells cultured in the complete culture medium without the incubation with AuNPRs/AuNPSs-MBA.

### Example 1

In this Example, the synthesis and surface modification of gold nanoparticles on gold nanorod supertructures (AuNPRs) is described.

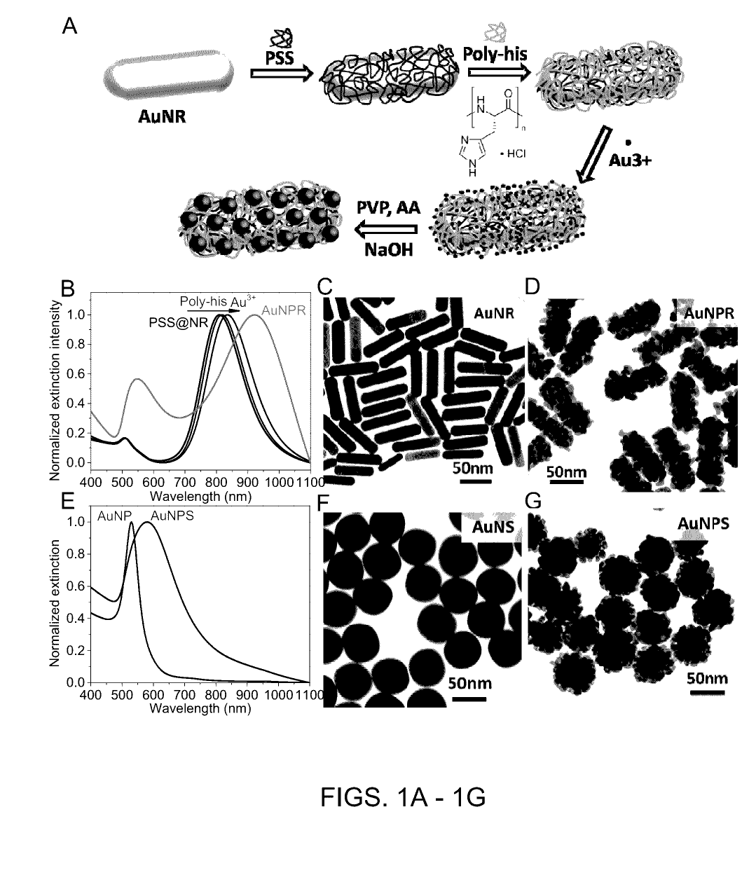

AuNPRs were synthesized by employing gold nanorods (AuNRs) as nanostructure cores. Anisotropic plasmonic superstructures, Au nanoparticles on rod (AuNPRs) were synthesized by coating Au nanorod (AuNR) cores with poly-L-histidine (poly-his), a biopolymer that enables the uniform nucleation of Au nanoparticles that form the nanoparticle satellites on the AuNR core (FIG. 1A).

First, 100 μl of aqueous poly-L-histidine (poly-his) solution (5 mg/ml) was added to 1 ml of PSS coated AuNRs (concentration adjusted to realize an extinction intensity of 2.0) followed by brief vortexing and incubation for 10 minutes. After centrifugation at 7,000 rpm for 10 minutes, the pellet was dispersed in nanopure water (18.2MΩ-cm). Immediately, 30 μl of aqueous HAuCl₄ solution (20 mM) was added to the above solution, followed by adjusting the pH of the reaction solution to 6.7 by the addition of 28 μl of aqueous NaOH solution (100 mM). After 3 minutes, 20 μl of aqueous ascorbic acid solution (1 M) as a reducing agent and 200 μl of aqueous polyvinylpyrrolidone solution (90 mM) as a capping agent were added respectively. Different amounts of HAuCl₄ (5-60 μl) was used to control the packing density of clusters grown on AuNR template. To 1 ml of once centrifuged AuNPRs, 80 μl of 2 mM methoxy polyethylene glycol thiol (SH-PEG) aqueous solution and 6 al of 10 mM pMBA ethanol solution were added subsequently, after 1 hour shaking. The above solution was centrifuged and dispersed in phosphate and acetate buffer from pH 5.0 to 9.0 to calibrate the intensity ratio of Raman bands 1394 cm⁻¹/1079 cm⁻¹ as a function of pH. The centrifuged PEGlated AuNPRs-pMBA was also dispersed in RPMI-1640 medium with 10% fetal bovine serum (FBS) for cellular experiments. Similarly, AuNPSs were synthesized by using 10/20 μl of 20 mM HAuCl₄ to 1 ml of PSS coated AuNSs with extinction 1.0.

Realization of AuNPR starts with the seed-mediated synthesis of AuNRs with a diameter of ˜15 nm and a length of ˜65 nm as illustrated in FIG. 1A and shown in TEM images in FIG. 1C. Subsequently, the AuNR were modified with a strong polyanion, namely, poly(styrenesulfonate) (PSS). The polyanion layer renders excellent stability to AuNR in a wide range of pH and ionic strength conditions, which allows for the subsequent biopolymer adsorption and Au nanoparticle formation into nanoparticle satellites. Following the removal of excess PSS from the AuNR solution, poly-his was adsorbed on AuNR through its strong affinity to Au and electrostatic interaction with negatively charged PSS. The presence of PSS and poly-his on the AuNR was confirmed using zeta-potential measurements and SERS (FIGS. 6A & 6B). For growing the Au nanoparticle satellites on AuNR, chloroauric acid (HAuCl₄), which serves as Au precursor, was introduced into the poly-his modified AuNR.

Adsorption of poly-his on AuNR and subsequent capture of Au³⁺ ions by imidazole groups of poly-his resulted in a red shift of 8.5 nm and 16.0 nm in the longitudinal localized surface plasmon resonance (LSPR) wavelength of AuNR, respectively (FIG. 1B). Subsequently, polyvinylpyrrolidone (as a capping agent) and ascorbic acid (as a mild reducing agent) were introduced into the reaction solution to result in the formation of nanoparticle satellite clusters on the AuNR surface.

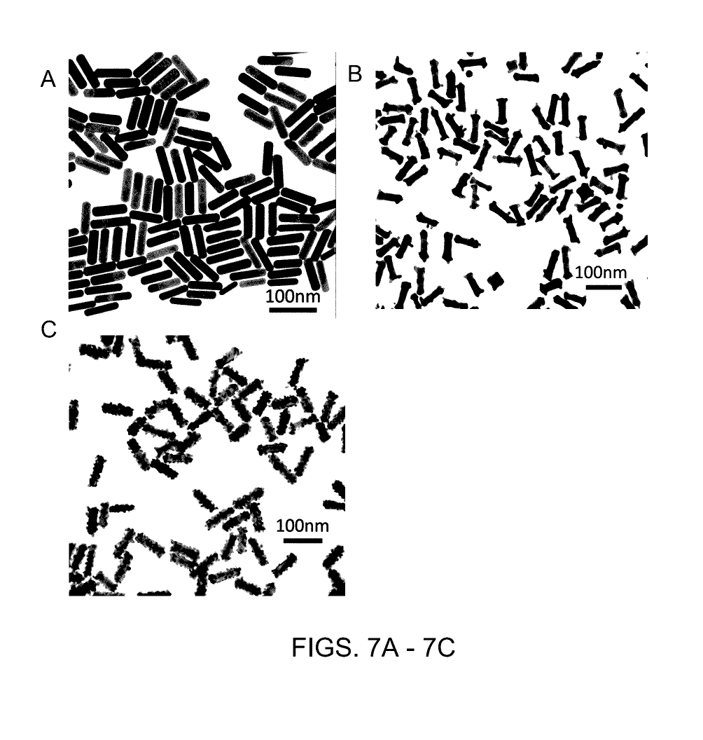

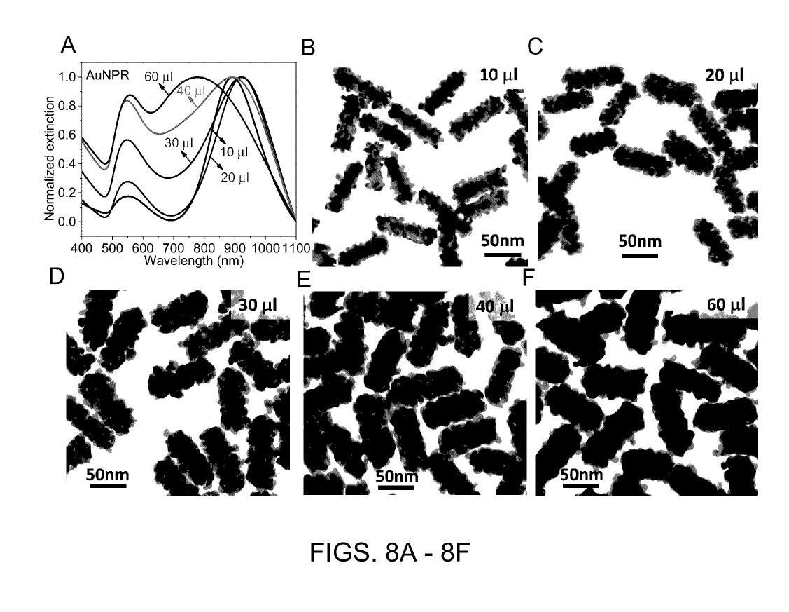

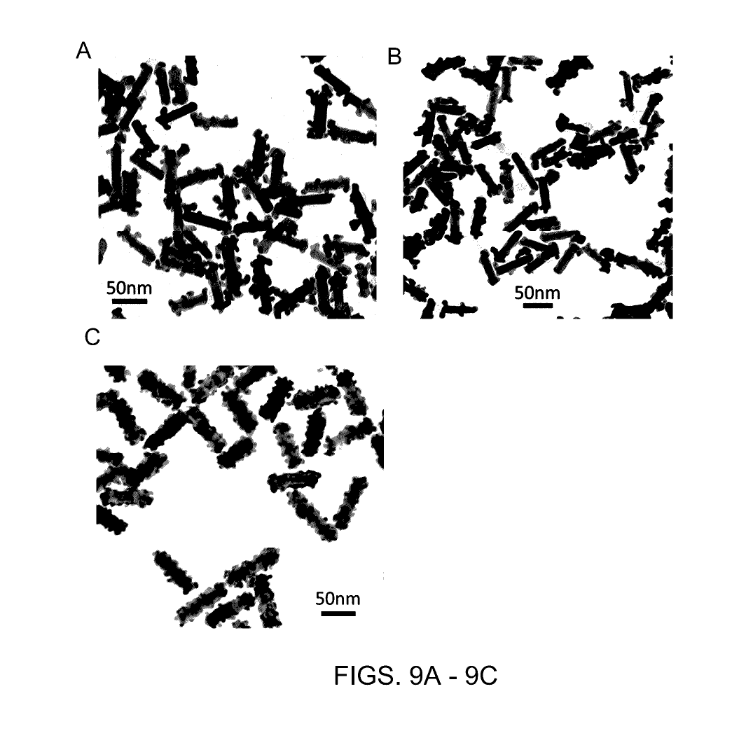

Plasmon coupling between the core and the nanoparticle satellites and the increased dimension of AuNR resulted in a red shift of 40.5 nm and 111.0 nm in the transverse and longitudinal LSPR wavelength (FIG. 1B). The strong affinity of poly-his to Au⁰, and Au³⁺ ions facilitates the uniform nucleation and growth of Au nanocrystals on the AuNR core as revealed by the TEM images (FIGS. 1C and 1D). In the absence of the poly-his coating, poor control over growth of Au nanoparticle satellites on PSS-coated AuNR was noted, which suggests the contribution of poly-his in the uniform growth of Au nanoparticle satellites on AuNR (compare FIGS. 7A & 7B). The size and areal density of gold nanoparticle satellites grown on AuNR core, which determine the optical properties of the superstructures, can be tuned over a broad range by varying the amount of Au precursor in the growth solution (FIGS. 8A-8F). The pH of the reaction solution also played an important role in the uniform nucleation of Au nanoparticle satellites on AuNR cores with an optimal pH being 6.4 (FIGS. 9A-9C).

Similar synthesis strategy was employed for other shape-controlled nanostructures such as on Au nanospheres (AuNS), which resulted in Au nanoparticle satellites (nanoparticles) on spheres (AuNPS) (FIGS. 1F and 1G). Following the growth of Au nanoparticle satellites, the LSPR wavelength of AuNS exhibited a red shift of 50.0 nm corresponding to the plasmon coupling between the core and nanoparticle satellites and increase in the diameter (FIG. 1E). The biotemplated synthesis approach demonstrated here serves as a universal method to realize size- and shape-controlled plasmonic superstructures.

### Example 2

In this Example, spacing formed between the nanoparticle satellites on AuNPR and AuNPS was analyzed.

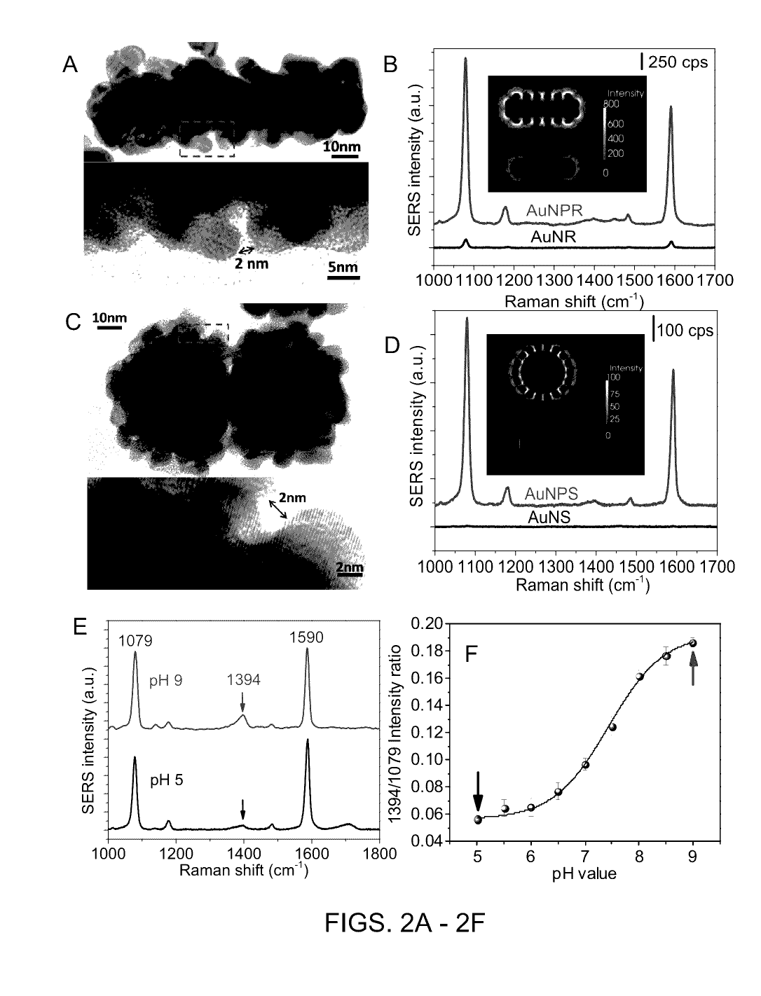

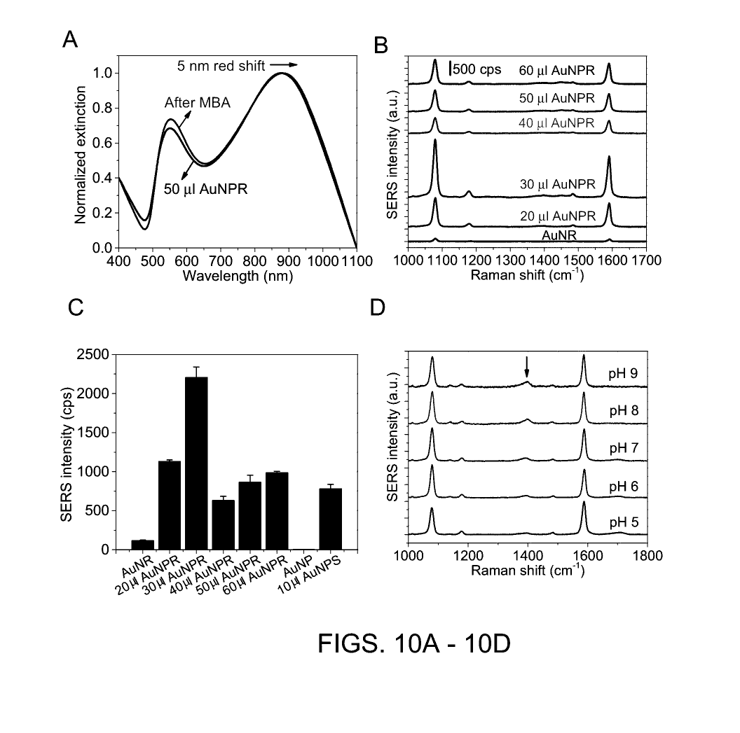

High-resolution transmission electron microscopy (HRTEM) images of AuNPR and AuNPS reveal sub-3 nm interstices formed between the nanoparticle satellites on AuNR and AuNS surface (FIGS. 2A and 2C). These interstices are open and accessible to surrounding solvent environment, enabling diffusion of Raman reporter molecules into the EM hotspots, and more importantly, facile sampling of the surrounding environment. Following the adsorption of Raman reporter, p-mercaptobenzoic acid (pMBA), SERS spectra collected from the superstructure solutions revealed strong SERS signals corresponding to the reporter molecules (FIGS. 2B, 2D and 10A). As noted earlier, the size and areal density of the nanoparticle satellites on the AuNR core, which determine the geometry of the EM hotspots, were found to significantly influence the SERS activity of the superstructures (FIGS. 10B & 10C). Compared to the corresponding cores, AuNPR and AuNPS exhibited approximately 20 and 200 times higher SERS intensity, respectively (FIGS. 2B and 2D). Finite-difference time-domain (FDTD) simulations confirmed the large enhancement of EM field in the interstices between the nanoparticle satellites (Insets of FIGS. 2B and 2D). Nanostructures with such highly developed morphology and built-in EM hotpots preclude the need for controlled aggregation or assembly of plasmonic nanostructures to achieve high SERS activity. Despite the weak plasmonic extinction in the NIR region, AuNPS exhibited significantly strong SERS signals with 785 nm excitation due to the EM hotspots formed between the nanoparticle satellites.

### Example 3

In this Example, pMBA was used as a pH sensitive Raman reporter to monitor the pH of the aqueous surroundings to demonstrate the functional molecular bioimaging capability of plasmonic superstructures by utilizing the accessible EM hotspots.

After confirming high SERS activity of plasmonic superstructures as discussed above, SERS spectra was collected from pMBA-modified AuNPRs dispersed in different pH buffers ranging from pH 5 to pH 9 with a 0.5 pH unit interval (FIGS. 2E and 10D). The SERS spectra from AuNPR show two strong Raman bands at 1079 cm cm⁻¹ and 1590 cm⁻¹, corresponding to the aromatic ring mode of pMBA. The ratio of the intensities of symmetric carboxylate stretching band (at 1394 cm cm⁻¹) and aromatic ring mode of pMBA (at 1079 cm⁻¹ or 1590 cm⁻¹) was found to increase with increasing pH due to the deprotonation of the carboxylate group. The measured pKa value of pMBA adsorbed on AuNPR surface is about 7.4, which is close to physiological pH and makes it an ideal Raman reporter for probing pH in biological applications. A pH calibration curve was obtained by plotting the intensity ratio of Raman bands 1394 cm⁻¹/1079 cm⁻¹ as a function of pH (FIG. 2F). Since the pH is correlated to the ratio of the intensity of two bands, the absolute intensity of the Raman bands, determined by the number of AuNPR in the focal volume, is not very important as long as the intensity of the two Raman bands is sufficiently high.

### Example 4

In this Example, intracellular pH imaging ability of AuNPRs was determined.

Individual plasmonic nanostructures as SERS probes have not been employed for spatiotemporal mapping of living cells to quantify intravesicular pH changes along endocytic pathways and exocytosis of internalized nanomaterials. Currently, pH in living cells is primarily quantified using fluorescent dyes that are plagued by photobleaching, low fluorescence quantum yield and narrow pH probing range. SERS, on the other hand, is highly attractive; for functional molecular bioimaging owing to numerous advantages including high sensitivity and specificity, excellent photostability, absence of interference from water, and high spatial resolution. However, only a few pH-sensitive SERS probes rely on the assemblies or aggregates of gold or silver nanoparticles to improve the brightness.

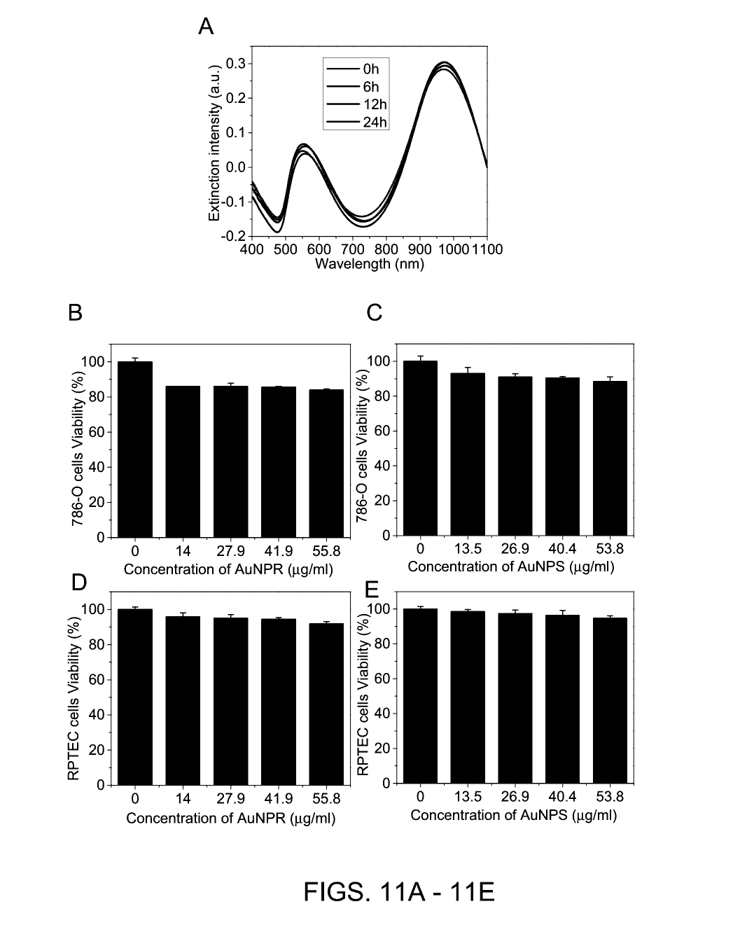

For demonstrating the intracellular pH imaging ability of AuNPRs, human renal adenocarcinoma cell line 786-O were used as a model cell line. To ensure the serum stability and biocompatibility of Au superstructures, the superstructure surface was coated with thiol-modified polyethylene glycol (SH-PEG), a non-toxic and hydrophilic polymer as a protective layer. The stability of AuNPRs were confirmed by monitoring the vis-NIR extinction spectra of PEGylated AuNPRs-pMBA at several time points following their dispersion in 10% fetal bovine serum (FBS) at 37° C. The extinction spectra of AuNPRs-pMBA showed ˜2 nm of red shift in longitudinal LSPR wavelength within the first 6 hours, corresponding to non-specific adsorption of serum proteins (FIG. 11A).

The biocompatibility of AuNPRs-pMBA was verified by performing (3-(4,5-dimethylthiazol-2-yl)-2,5-diphenyltetrazolium bromide (MTT) cell viability assay (FIGS. 11B-11E). The concentration of superstructures at which the cell viability was at least 85% or higher (compared to the control cells) was used for subsequent studies.

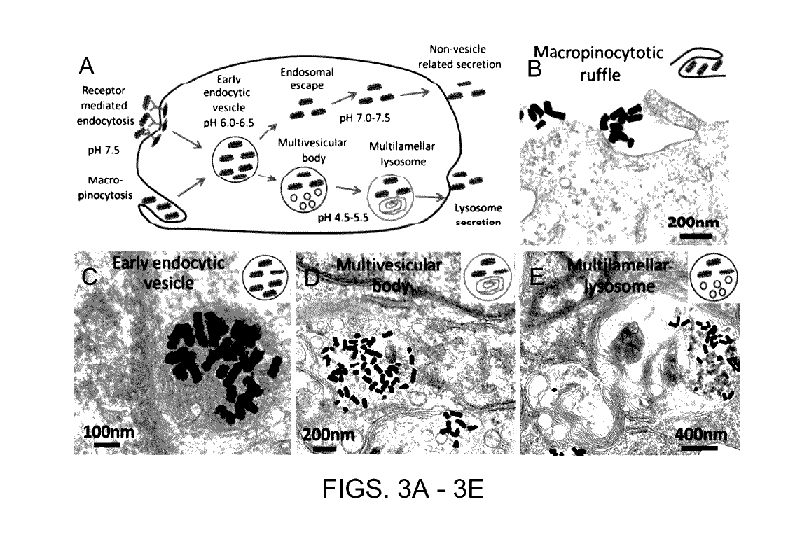

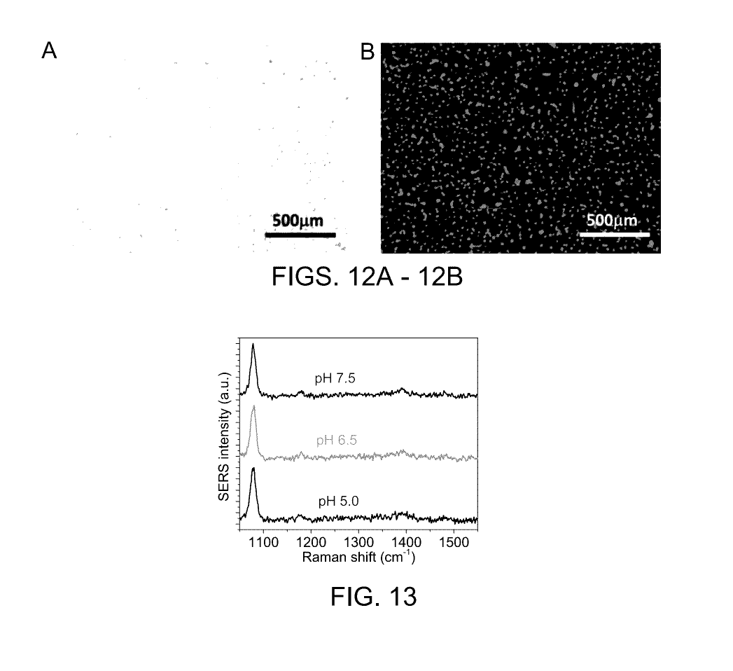

786-O cells seeded on a quartz substrate were incubated with PEGylated AuNPRs-pMBA to facilitate internalization, followed by the removal of free AuNPRs. The uptake of AuNPRs by 786-O cells was facilitated by micropinocytosis and receptor-mediated endocytosis due to non-specific adsorption of serum proteins on AuNPR surface (FIGS. 3A, 3B). After internalization, AuNPRs encapsulated inside intracellular compartments go through an endocytic pathway with a characteristic acidification profile from pH 6.0-6.5 in early endocytic vesicles to pH 4.5-5.5 in multivesicular bodies (MVBs) and multilamellar lysosomes (MLs). Some of the internalized AuNPRs escaped from endosomes to cytosol (pH 7.0-7.5) and directly exocytose from cells (FIG. 3A). TEM images of ˜100 nm thick cell sections revealed uptake of AuNPRs by micropinocytosis and AuNPRs-trafficking vesicles in a size range of 0.5-2.0 μm, including small early endocytic vesicles, large MVBs containing many small luminal vesicles, and MLs with characteristic membrane whorls enclosed (FIGS. 3B-3E). The internalized AuNPRs preserved the core-satellite superstructure, which allows for their high SERS activity (FIGS. 3C, 12A, & 12B).

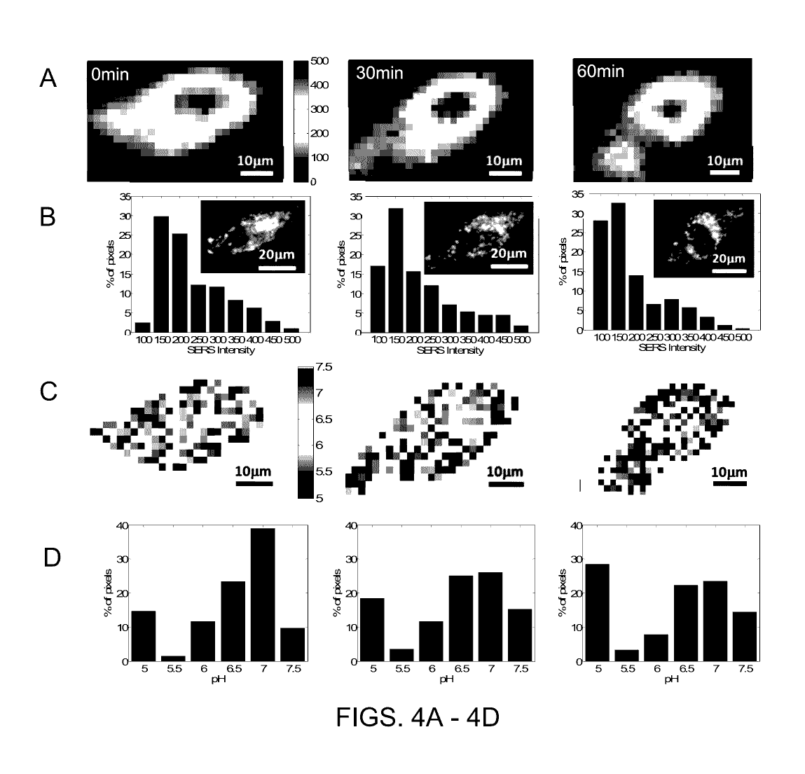

Raman imaging was performed using a live cell chamber under a confocal Raman microscope after locating the cells under dark-field illumination. The intensity maps of Raman band at 1079 cm⁻¹ obtained at 0, 30 and 60 minutes using a 785 nm laser as excitation source enabled the clear delineation of the cells shape (FIG. 4A). Dark-field optical images of the cells revealed the large Rayleigh scattering from the internalized and cell surface-bound AuNPRs (inset images of FIG. 4B). Raman intensity histograms obtained from intensity maps at different time points showed ˜10% mean intensity drop after 30 minutes duration and ˜20% drop after 60 minutes (FIG. 4B). The intensity drop corresponds to the ˜20% exocytosis of AuNPRs within 1 hour. The exocytosis fraction was validated using a conventional method to quantify the change of gold content after exocytosis by inductively coupled plasma mass spectroscopy (ICP-MS). ICP-MS measurements indicated an average uptake of nearly 7.1×10³ AuNPRs per cell after 8 hours of incubation, which is slightly higher than reported values probably due to the differences in size, shape, surface coating, initial concentration, incubation time and sedimentation effect of nanoparticles and cell lines. ICP-MS results also showed ˜20% exocytosis of AuNPRs over 1 hour of incubation in complete medium at 37° C., confirming the exocytosis fraction calculated from Raman intensity maps, which is also in agreement with reported typical exocytosis fraction.

Intracellular pH map was obtained by comparing the intensity ratio of Raman bands at 1394 cm⁻¹ and 1079 cm⁻¹ at each pixel with a pH calibration curve shown in FIG. 2F. Each pixel was color-coded using MATLAB®, with red to blue representing the progressive transition from physiological pH to more acidic values (FIG. 4C). The physiological pH 7.0-7.5 corresponds to AuNPRs on the cell surface or at a very early stage of internalization that in the close proximity to the cell membrane, or those that escaped from endosomes. The time-dependent pH histograms showed the decrease in the fraction of physiological pH 7.0-7.5 from 49% to 38% after 1 hour, suggesting that some surface bound AuNPRs proceed to early endosomes and the AuNPRs that escaped from endosomes are exocytosed (FIG. 4D). The fraction of pH 6.0-6.5 decreased from 35% to 30%, indicating the trafficking of AuNPRs from early endosomes to more acidic MVBs and MLs. The trafficking was also confirmed by the increase in the fraction of pH 5.0-5.5 from 16% to 32% by 1 hour, which visualizes the intravesicular pH drops in the endosomal maturation process. Owing to the accessible electromagnetic hotspots that enable facile sample of the surrounding environment, the plasmonic superstructures of the present disclosure provide excellent candidates for functional molecular bioimaging.

### Example 5

In this Example, the photothermal efficacy of plasmonic superstructures comprised of AuNR cores and plasmonic superstructures comprised of AuNS cores was determined.

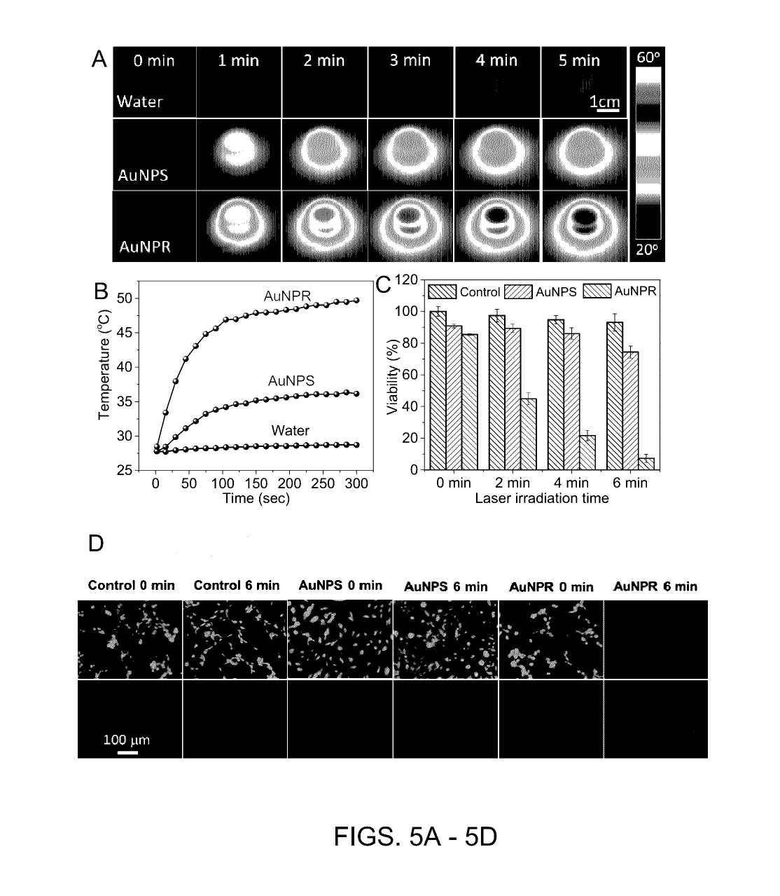

The temperature rise of AuNPR and AuNPS solutions upon irradiation with 808 nm laser (at a power density of 0.3 W/cm²) was monitored using an infrared camera (FIG. 5A, 5B). The temperature of the superstructure solutions exhibited significant increase within the first 100 seconds (S) of irradiation followed by either small increase or stabilization for subsequent irradiation. At t=300 S, AuNPR exhibited nearly 24° C. rise in temperature while AuNPS exhibited significantly smaller rise in temperature (˜8° C.) under identical conditions. The significantly higher rise in temperature of AuNPR solution compared to AuNPS solution can be rationalized from the vis-NIR extinction spectra of these nanostructures, which demonstrate the significantly higher absorbance of the AuNPR at 808 nm compared to AuNPS at the similar concentration of ˜55 g/ml Au atoms.

### Example 6

In this Example, the photothermal therapeutic ability of plasmonic superstructures was determined.

To determine the photothermal therapeutic ability of the plasmonic superstructures in vitro, 786-O cells at 90% confluence in 24 well plates were incubated with AuNPR and AuNPS to facilitate internalization of the nanostructures. Following the removal of free plasmonic superstructures, the cells were irradiated with 808 nm laser for different durations (0-6 minutes) followed by incubation at 37° C. and 5% CO₂ for 18 hours.

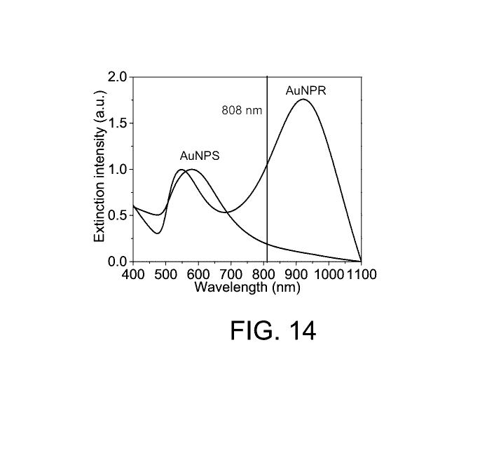

Cell viability quantified by MTT assay indicated significantly higher cell death for different irradiation times for AuNPR compared to that of AuNPS, which is in complete agreement with the higher photothermal efficiency of AuNPR compared to AuNPS (FIG. 5C). Whereas the viability of cells incubated with AuNPS remained at 80% even after irradiation for 6 minutes, the viability of cells incubated with AuNPR dropped to 10% for the same irradiation conditions. These results were further confirmed by a live/dead cell assay performed after 6 minutes of irradiation for cells incubated with AuNPS and AuNPR (FIG. 5D). Presence of strong green fluorescence (corresponding to the live cells) and absence of red fluorescence (corresponding to the dead cells) was noted for cells incubated with AuNPS following the laser irradiation whereas the inverse was noted for cells incubated with AuNPR. These results demonstrate that AuNPRs, which exhibit strong SERS activity and absorbance in the NIR wavelengths, can serve as multifunctional theranostic probes that can be employed to image and treat cancer. On the other hand, AuNPS, which exhibit weak absorbance but strong SERS activity using near infrared excitation can efficiently decouple imaging from unwanted photothermal heating (FIGS. 2D and 14).

The results presented herein demonstrate a simple and universal approach to synthesize plasmonic superstructures comprised of a shape-controlled nanostructure core and densely packed nanoparticle satellites. The biotemplated approach demonstrated here can be extended to any nanostructure to obtain superstructures with desired optical properties. As opposed to conventional structural SERS imaging probes, the core-satellite plasmonic superstructures with accessible electromagnetic hotspots of the present disclosure can serve as functional contrast agents to sense and report a specific (bio)chemical stimulus or molecular process in complex biological milieu. Furthermore, in these EM hotspot-dominated plasmonic superstructures, the SERS activity can be decoupled from LSPR wavelength making them ideal for unperturbed bioimaging. Additionally, the plasmonic superstructures can be designed to serve as imaging and therapeutic agents by rational choice of the size and shape of the nanostructure cores.

All of the compositions and/or methods disclosed and claimed herein may be made and/or executed without undue experimentation in light of the present disclosure. While the compositions and methods of this disclosure have been described in terms of the embodiments included herein, it will be apparent to those of ordinary skill in the art that variations may be applied to the compositions and/or methods and in the steps or in the sequence of steps of the method described herein without departing from the concept, spirit, and scope of the disclosure. All such similar substitutes and modifications apparent to those skilled in the art are deemed to be within the scope and concept of the disclosure as defined by the appended claims.

What is claimed is:

1. A plasmonic superstructure comprising:
a nanostructure core;
a polymer coating the nanostructure core; and
a plurality of nanoparticle satellites coupled to the nanostructure core.

2. The plasmonic superstructure of claim 1, further comprising a polyanion layer.

3. The plasmonic superstructure of claim 2, wherein the polyanion layer is selected from the group consisting of poly(styrenesulfonate); poly(acrylic acid), alginate, and combinations thereof.

4. The plasmonic superstructure of claim 1, wherein the nanostructure core is selected from the group consisting of a nanorod; a nanosphere; a nanocube; a nanobipyramid; a nanostar; and combinations thereof.

5. The plasmonic superstructure of claim 1, wherein the polymer is selected from the group consisting of a biopolymer, a synthetic polymer, and combinations thereof.

6. The plasmonic superstructure of claim 5, wherein the biopolymer is selected from the group consisting of poly-L-histidine, poly(tyrosine), and combinations thereof.

7. The plasmonic superstructure of claim 5, wherein the synthetic polymer is selected from the group consisting of poly(allylamine hydrochloride), poly (2-vinyl pyridine), and combinations thereof.

8. The plasmonic superstructure of claim 1, further comprising a protective layer.

9. The plasmonic superstructure of claim 8, wherein the protective layer comprises a hydrophilic polymer.

10. The plasmonic superstructure of claim 9, wherein the hydrophilic polymer comprises thiol-modified polyethylene glycol, amine-terminated polyethylene glycol, carboxyl-terminated polyethylene glycol and combinations thereof.

11. The plasmonic superstructure of claim 1, further comprising a Raman reporter.

12. The plasmonic superstructure of claim 11, wherein the Raman reporter is p-mercaptobenzoic acid.

13. The plasmonic superstructure of claim 1, wherein the plurality of nanoparticle satellites comprises an interstices distance between nanoparticle satellites of about 3 nm or less.

14. A method of preparing a plasmonic superstructure, the method comprising:
preparing a nanostructure core;
incubating the nanostructure core with a polymer solution to coat the nanostructure core with the polymer;
incubating the nanostructure core with a metal nanoparticle satellite precursor solution, wherein a plurality of nanoparticles form a plurality of nanoparticle satellites coupled to the nanostructure core to form the plasmonic superstructure.

15. The method of claim 14, further comprising incubating the nanostructure core with a polyanion solution.

16. The method of claim 15, wherein the polyanion is selected from poly(styrenesulfonate); poly(acrylic acid), alginate, and combinations thereof.

17. The method of claim 14, wherein the polymer is selected from the group consisting of a biopolymer, a synthetic polymer, and combinations thereof.

18. The plasmonic superstructure of claim 17, wherein the biopolymer is selected from poly-L-histidine, poly(tyrosine), and combinations thereof.

19. The plasmonic superstructure of claim 17, wherein the synthetic polymer is selected from poly(allylamine hydrochloride), poly (2-vinyl pyridine), and combinations thereof.

20. The method of claim 14, further comprising preparing a protective layer.
